(19) United States
(12) Patent Application Publication
(10) Pub. No.: US 2001/0041683 A1
(43) Pub. Date: Nov. 15, 2001

(54) Cocoa sphingolipids, cocoa extracts containing sphingolipids and methods of making and using same

(76) Inventors: Harold H. Schmitz, Branchburg, NJ (US); Sheryl Lazarus, Centerville, OH (US); John F. Hammerstone, Nazareth, PA (US)

Correspondence Address: Clifford Chance Rogers & Wells LLP, 200 Park Avenue, New York, NY, 10166-0153, US

(21) Appl. No.: 09/801,494

(22) Filed: Mar. 8, 2001

Related U.S. Application Data
(60) Provisional application No. 60/187,950, filed on Mar. 9, 2000.

(51) Int. Cl. A61K 31/7008; A61K 31/715; A61K 31/685; A61K 9/68

(52) U.S. Cl. 514/54; 514/78; 514/42; 424/440

(57) ABSTRACT

The invention relates to cocoa sphingolipids, cocoa extracts containing cocoa sphingolipids, and compositions containing cocoa sphingolipids and/or metabolic derivatives thereof. The invention also relates to methods of isolating and purifying sphingolipids and methods of using the cocoa sphingolipids and/or metabolic derivatives thereof.

This application claims priority under 35 U.S.C. § 119 from the U.S. Provisional Application Ser. No. 60/187,950, filed Mar. 9, 2000, which application is hereby incorporated herein by reference in its entirety.

### FILED OF THE INVENTION

The invention relates to cocoa sphingolipids, cocoa extracts containing cocoa sphingolipids, and compositions containing cocoa sphingolipids and/or metabolic derivatives thereof. The invention also relates to methods of isolating and purifying sphingolipids and methods of using the cocoa sphingolipids and/or metabolic derivatives thereof.

### BACKGROUND OF THE INVENTION

In the late nineteenth century, sphingolipids were discovered in the mammalian brain by J. L. W. Thudichum. It is now known that sphingolipids are not limited to the brain; they can be found in all eukaryotic and some prokaryotic organisms (Merrill et al., Symposium: Animal Diets for Nutritional and Toxicological Research, pp. 830S-33S, Am. Soc. Nutr. Sci. 1997, Witaker, Phytochem., 42:627-632, 1996; Ohnishi et al., Biochimica Biophysica Acta, 752: 416-422; 1983; Ohnishi and Fujino, Lipids, 17:803-810 (1982); Laine and Renkonen, Biochem., 13:2837-43; 1974; Walter et al., 36:795-97; 1971)).

Sphingolipids are compounds having a long-chain base (often referred to as sphingoid base backbone), which may be saturated or unsaturated. The sphingoid base is usually bonded, via its amino group, to a fatty acid. The fatty acid has 16-30 carbons and occasionally may contain an a-hydroxy group. An example of a sphingolipid is represented in FIG. 1. More complex sphingolipids may contain, as shown in FIG. 1, a polar headgroup at position 1 of the sphingoid base. Over 70 sphingoid bases, dozens of amide-linked fatty acids and over 300 headgroups have been thus far identified (Merrill, 1997).

Sphingolipids are both structural and functional lipids. Their physical properties (such as high phase transition temperature) affect the properties of membranes and lipoproteins, and they are critical components of water barrier of skin. They serve as ligands for extracellular matrix proteins and receptors on neighbouring cells, as well as for enteric bacteria and viruses. Sphingolipids are also important in cell regulation as modulators of growth factor receptors and as second messengers for a number of agonists, including tumor necrosis factor-α, interleukin-1β, nerve growth factor and 1α,25-dihydroxyvitamin D3 (Merrill 1997). The intracellular targets of sphingolipids include protein kinases and phosphoprotein phosphatases. Sphingolipids modulate calcium levels and are involved with other signal transduction pathways (Schmelz and Merrill, Nutrition, 14:717-19, 1998). Cellular functions of sphingolipids have been described, for example, in Merrill 1997; Vesper et al., J. Nutr. 129:1239-1250, 1999.

When ingested, sphingolipids are metabolized in all regions of the intestinal tract and the metabolic products are taken up by the cells (Schmelz and Merrill, 1998). Administration of sphingomyelin by gavage has shown that about 10% of undigested sphingomyelin reaches the colon where it is metabolized by intestinal enzymes to release ceramide and free sphingoid bases (Schmeltz et al., J. Nutr, 124:702, 1994).

Despite considerable information about sphingolipids and their biological function, very few studies concentrated on the analysis of the types and amounts of sphingolipids in foods (Merrill, 1997). Some information exists for foods of plant origin. Thus far, sphingolipids have been identified and quantified in some fruits (e.g., apple, orange, banana) and grains (e.g., wheat, rice, soybeans) (Vesper et al., 1999). The amount of sphingolipids varies from less than about 0.1 μmol/g fresh weight in apples (Hitchock and Nichols, Plant Lipid Biochemistry, Academic Press, London, 1971) to about 2 μmol/g dry weight in soybeans (Ohnishi and Fujino, Lipids, 17:803-810, 1982). The sphingolipids identified in plants are mainly cerebrosides (mono- and oligohexosylceramides, i.e., they contain a mono- or oligohexosyl headgroup) (Merrill, 1997, Vesper et al., 1999).

There remains a need in the art to identify, isolate and quantify the sphingolipids in plants that are used in the human diet. Sphingolipids in cocoa have now been isolated, quantified and characterized.

### SUMMARY OF THE INVENTION

The invention relates to cocoa sphingolipids, cocoa extracts containing cocoa sphingolipids, and compositions, such as foods, dietary supplements and pharmaceutical products containing cocoa sphingolipids and sphingolipid-containing extracts. The invention also relates to methods of making and using cocoa sphingolipids.

In one aspect, the invention relates to cocoa extracts containing sphingolipids as well as extracted and purified cocoa sphingolipids and metabolic derivatives thereof.

In another aspect, the invention relates to compositions, for example, foodstuffs, dietary supplements and pharmaceutical products comprising the cocoa extracts and/or purified sphingolipids and their metabolic derivatives.

In a further aspect, the invention relates to a method of extracting and purifying sphingolipids from cocoa or any other sphingolipid-containing material.

In yet another aspect, the invention relates to methods of using sphingolipid containing cocoa extracts, cocoa sphingolipids and metabolic derivatives thereof, for example, for controlling cell proliferation, treating cancer, inhibiting protein kinase-C and mediating action of tumor necrosis factor α, interleukin 1β, and nerve growth factor.

### BRIEF SUMMARY OF THE DRAWINGS

FIG. 1 illustrates the basic structure of sphingolipids.

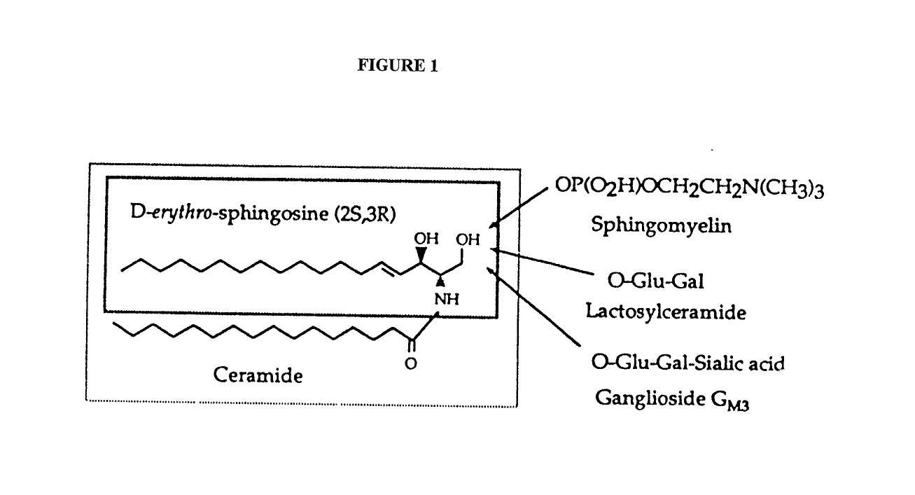

FIG. 2 shows MS spectra of certain cocoa sphingolipids.

FIG. 3 shows MS spectra of certain cocoa sphingolipids.

FIG. 4 shows MS spectra of certain cocoa sphingolipids.

### DETAILED DESCRIPTION OF THE INVENTION

All patents, patent applications and references cited herein are hereby incorporated by reference in their entirety. In case of any inconsistency, the present disclosure governs.

The cocoa sphingolipids are cerebrosides with a sugar headgroup, preferably hexosylceramides, more preferably, glycosylceramides (“GlcCer”). Cocoa sphingolipids are composed of a sphingoid base, a fatty acid and a sugar headgroup. The headgroup of the cocoa ceramide may be a hexose, for example glucose, mannose, galactose or inositol, or a pentose. At least one sugar is present but the number of sugar units may be greater than one. More than one type of sugar may be present in a single sphingolipid molecule, for example more than one hexose may be present in the oligohexosyl headgroup. Cocoa GlcCer contains glucose as a headgroup.

The sphingoid base is composed of 18 carbon atoms, one or two double bonds and two or three hydroxyl groups. The double bonds are located in the sphingoid base at position Δ4 (i.e., between carbon atoms 4 and 5) and/or position Δ8 (i.e., between carbon atoms 8 and 9). The hydroxyl groups are located at positions 1 and 3, when two are present in the sphingoid base and at positions 1,3, and 4, when three are present in the sphingoid base. The fatty acid component of the sphingolipid is composed of from about 16 to about 28 carbon atoms and may contain an a-hydroxyl group at position 2.

Examples of cocoa sphingolipids are: d18:2^{Δ4,Δ8}/c16:0; d18:2^{Δ4,Δ8}/c18:0; d18:2^{Δ4,Δ8}/c20:0; d18:2^{Δ4,Δ8}/h16:0; d18:2^{Δ4,Δ8}/h18:0; d_{18:2}^{Δ4,Δ8}/h20:0; d18:2^{Δ4,Δ8}/h22:0; d_{18:2}^{Δ4,Δ8}/24:0; t18:1^{Δ8}/h20:0; t_{18:1}^{Δ8/}h22:0; t18:1^{Δ8}/h24:0; t_{18:1}^{Δ8}/h26:0; and t18:1^{Δ8}/h28:0.

Exemplary sphingolipids are represented by the following formulas:

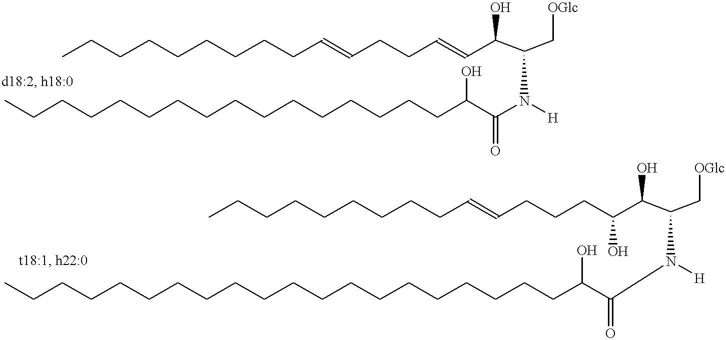

The nomenclature used herein is generally known and accepted in the art. In summary, the first set of numbers identifies the sphingoid base, and the second set of numbers identifies the fatty acid. Referring to sphingoid bases, “d” means 1,3, dihydroxyl, and “t” means 1,3,4, trihydroxyl; the first number (18) refers to the number of carbon atoms in the sphingoid base, and the second number (1 or 2) refers to the degree of unsaturation, i.e., the number of double bonds. Thus, d18:2 is sphingediene and t18:1 is hydroxysphingenine. Referring to the fatty acids, “c” means there is no α-hydroxyl group and “h” means presence of an α-hydroxyl group; the first number indicates the number of carbon atoms in the chain and the second number refers to the number of double bonds. Thus, h16:0 is α-hydroxypalmitic acid, h18:1 is α-hydroxystearic acid, h22:0 is α-hydroxybehenic acid, h24:0 is α-hydroxylignoceric acid, c16:0 is palmitic acid, c18:0 is stearic acid and c20:0 is arachidonic acid.

The cocoa sphingolipids may be isolated from cocoa beans, cocoa mass, partially or fully defatted cocoa solids, e.g. cocoa powder, and/or chocolate liquor as described herein. In fact, any “cocoa material,” i.e., product derived from cocoa bean, with the exception of cocoa butter may be used. Cocoa butter is not preferred because it contains minimal quantities of sphingolipids. The cocoa sphingolipids may also be synthetically prepared. Sphingosine or phytosphingosine bases may be used as starting materials in the synthesis of sphingolipids. Such methods are generally known in the art and are described, for example, in Schmidt et al., Carbohydrate Research, 174:169-179 (1988); U.S. Pat. No. 5,780,441 to Higa et al., Schmelt et al., Nutr. Cancer, 28:81 (1997); U.S. Pat. No. 5,677,337 to Wei et al. It is also known to prepare sphngolipids in microorganisms as described, for example, in U.S. Pat. No. 5,958,742.

Cocoa extracts which contain sphingolipids are referred to herein as “sphingolipid-containing cocoa extracts”. As indicated above, the extracts are prepared from any cocoa material. The cocoa extracts may contain at least about 25%, preferably at least about 50%, more preferably at least 75%, and most preferably at least about 90% of cocoa sphingolipids. The sphingolipid-containing cocoa extract is substantially free of cocoa polyphenols, such as cocoa procyanidins. As used herein, “substantially free” means containing insignificant, trace, and/or minimal quantities of a non-sphingolipid compound (e.g. cocoa polyphenol). In one embodiment, the sphingolipid-containing extract is free of cocoa polyphenol (or procyanidin), i.e., it does not contain detectable quantities of cocoa polyphenols (or procyanidins). The extract may also be substantially free of non-polar lipids, i.e., non-polar lipids may be present in minor amounts.

In one embodiment, the cocoa extract contains at least one substantially pure cocoa sphingolipid. As used herein, the phrase “substantially pure” means the highest degree of purity that can be achieved using purification methods known in the art.

In another embodiment, the cocoa extract is prepared by a process including the steps of extracting polar lipids from the cocoa material, subjecting the extract to base hydrolysis, and optionally removing non-polar lipid contaminants and/or the products of base hydrolysis. In another embodiment, the cocoa extracts are prepared according to the purification steps shown in Example 1.

The sphingolipid-containing cocoa extract may compromise at least one GlcCer. For example, at least one of the following cocoa sphingolipids may be present: d18:2^{Δ4,Δ8}/c16:0; d18:2^{Δ4,Δ8}/c18:0; d18:2^{Δ4,Δ8}/c20:0; d18:2^{Δ4,Δ8}/h16:0; d18:2^{Δ4, Δ8}/h18:0; d_{18:2}^{Δ4,Δ8}/h20:0; d18:2^{Δ4,Δ8}/h22:0; d18:2^{Δ4,Δ8}/24:0; t18:1^{Δ8}/h20:0; t18:1^{Δ8}/h22:0; t18:1^{Δ8}/h24:0; t_{18:1}^{Δ8}/h26:0; and t_{18:1}^{Δ8}/h28:0. In one embodiment, the extract comprises the GlcCers in the following order of prevalence:

d18:2,h18:0>d18:2,h16:0=t18:1,h22:0=t18:1,h24:0>d18:2,h16:0.

The invention further relates to isolated cocoa sphingolipids and metabolic derivatives thereof. As used herein “isolated” means sufficiently separated from its original environment. For example, isolated so that there are less than 25% of the original components, preferably less than 50% and most preferably less than 75% of the original components. Sphingolipids may be isolated from plant material or from synthetic preparations. They may be substantially pure. For example, a substantially pure compound may be obtained using High Performance Liquid Chromatography (HPLC). Both reverse-phase and normal HPLC may be used.

As used herein, a “metabolic derivative” of the cocoa sphingolipid is any compound which is produced when the sphingolipid is hydrolyzed. Metabolic derivatives obtained through in vivo hydrolysis, metabolism or digestion in a mammalian intestine, as well as those obtained in vitro by chemical hydrolysis are included herein. Examples of such metabolic derivatives are ceramides, sphingoid bases (e.g. 4, 8 sphingadiene, 4-hydroxy-8-sphingenine, sphinganine), sphingoid bases substituted at position 1 by a phosphate (e.g. 4, 8 sphingadiene 1-phosphate), fatty acids and glucose.

In one embodiment, a metabolic derivative of the cocoa sphingolipid has at least one of the following properties: altering the activity of protein kinases (including protein kinase C), inducing apoptosis or cellular differentiation, altering cell growth, mediating actions of tumor necrosis factor α, interleukin 1β, nerve growth factor, and 1α,25-dihydroxyvitamin D₃, inhibiting retinoblastoma protein dephosphorylation, activating phospholipase C/inhibiting phosphatidic acid phosphatase, altering diacylglycerol and cholesterol metabolism, and mobilizing Ca²⁺ from intracellular stores. As used herein, “altering” means in any way changing the state, activity or function of a molecule or cell from the one existing before the cocoa extract, cocoa sphingolipid, or the metabolic derivative thereof was added. For example, the change may be inhibition, activation, stimulation, etc. Examples of these metabolic derivatives are ceramides, sphingoid bases (e.g., 4, 8 sphingadiene, 4-hydroxy-8-sphingenine, sphinganine), and sphingoid bases substituted at position 1 by a phosphate group (e.g., 4, 8 sphingadiene 1-phosphate).

In a preferred embodiment, the useful metabolic derivative of the cocoa sphingolipid is as described above but excluding glucose or a fatty acid.

Cocoa extracts comprising cocoa sphingolipids can be obtained using the following procedure. Crushed cocoa beans, cocoa mass, and/or cocoa solids may be used as a stating material (hereinafter “cocoa material”). The cocoa material may be prepared as generally known in the art and as described, for example, in Industrial Chocolate Manufacture and Use, 2d ed., Ed. S. T. Beckett, Blackie Academic Professional, London, 1994. For example, the cocoa powder can be prepared by freeze-drying cocoa beans, depulping and dehulling the freeze-dried cocoa beans and grinding the dehulled beans. The cocoa material may be fully or partially defatted and may be fermented, underfermented or unfermented.

The method for extracting cocoa sphingolipids comprises the step of extracting polar lipids from the cocoa material to obtain a crude extract. The crude extract may be subjected to base hydrolysis primarily to cleave glycerolipids. Optionally, to increase the purity of extracted sphingolipids, i.e., to enrich for cocoa sphingolipids, a step of removing residual non-polar lipids (e.g. fatty acids) from the crude extract prior to base hydrolysis may be used. The term “residual non-polar lipids” refers to non-polar lipids extracted in small amounts together with polar components due to the limitations of the extraction procedure. In another optional step, subsequent to base hydrolysis, the cleaved glycerolipids (obtained upon base hydrolysis) may be removed from the extract together with other non-lipid polar components.

The step of extracting polar lipids from the cocoa material may be performed using an organic solvent such as a short-chain alcohol such as butanol, isopropanol, methanol, or a chloroform/methanol combination. More than one extraction step may be performed, with the same or different organic solvent, and the crude extracts may then be combined for purification. The solvent may be used heated or hot, for example, at least at 50° C. for about 5-10 minutes, preferably at least at 75° C. for the same time. The solvents may also be used at their boiling points.

The optional step of removing residual non-polar lipids, such as fatty acids, comprises at least one extraction with an alkane, such as hexane, ethyl ether, petroleum ether, or the like. The solvent extraction, e.g., hexane extraction, may be followed by a methanol wash.

The step of subjecting the crude cocoa sphingolipid extract to base hydrolysis is carried out by admixing the extract and the base in order to cleave glycerolipids present in the crude extract. Any base may be used, for example potassium hydroxide or sodium hydroxide. The base may be neutralized, as is well known in the art, for example, using hydrochloric acid mixed with methanol.

The step of removing the products of the base hydrolysis and/or the non-lipid polar components is carried out by extraction at least once with a chloroform/water mixture, for example a 2:1 mixture. In this extraction, the chloroform removes the products of the base hydrolysis and the water removes the polar non-lipid components such as the sugars and amines.

The sphingolipid-containing cocoa extract obtained following the above steps may be further purified and individual cocoa sphingolipids may be separated by HPLC, e.g. normal phase HPLC.

In one embodiment, the cocoa sphingolipids are extracted according to the following procedure. The cocoa material is extracted with hot butanol, preferably twice, and the crude extract, which primarily contains polar lipids, is removed and saved. Some non-polar contaminants such as fatty acids may be present in this extract. The cocoa material is then extracted, preferably twice, with a chloroform:methanol:water mixture to extract any remaining polar lipids. The ratio among these solvents in the mixture can be determined by a person of skill in the art keeping in mind the objective to extract highly polar lipids. For example, a 1:2:0.8 chloroforrn:methanol:water ratio may be used. Alternatively, as little methanol as in a 4:1 chloroforrn:methanol (without water) ratio and up to as much methanol as in a 1:2 chloroform:methanol ratio (with or without water) may be used. The chloroform/methanol and/or chloroform/methanol/water extracts are then combined with the butanol extract and dried. The dried crude sphingolipid-containing extract is reconstituted and extracted with hexane/methanol to separate non-polar lipids from polar lipids, which include sphingolipids. The hexane phase is separated, then extracted with methanol and dried. The dried cocoa extract is then reconstituted and subjected to base hydrolysis in the presence of potassium hydroxide or sodium hydroxide. The hydrolyzed extract is neutralized with, for example, hydrochloric acid in methanol and dried. To the extent that insignificant quantities of cocoa polyphenols are present in the crude extract, these compounds are completely eliminated during the step of base hydrolysis. The dried extract is then reconstituted and subjected to another extraction. A chloroform extraction, preferably three times, may be used. A 2:1 ratio of chloroform:water is suitable for this extraction. Hexane may be substituted for the chloroform at this stage should there be a need to avoid the use of chlorinated solvents. The chloroform extract is then dried and may be used or further purified and/or separated using chromatography, for example HPLC. Normal phase HPLC with a silica column, preferably amine column and most preferably diol column may be used. A mobile phase gradient from 97% acetonitrile/2% methanol to 67% methanol: 15% water: 18% butanol may be used.

The invention also relates to an improved method of extracting and purifying sphingolipids from any sphingolipid-containing material including any plant material. The method is particularly useful for eliminating fatty acids that may interfere with the extraction and purification processes. The method comprises the steps of extracting polar lipids from the material to obtain a crude extract, purifying the extract by removing residual non-polar lipids (e.g. fatty acids), subjecting the extract to base hydrolysis, and optionally further purifying the extract or separating individual sphingolipids. These steps may be performed as described above.

Compositions comprising sphingolipid-containing cocoa extracts, cocoa sphingolipids, metabolic derivatives thereof and any combinations thereof are also within the scope of the invention. In one embodiment, sphingolipid-containing cocoa extracts, cocoa sphingolipids, and/or metabolic derivatives thereof are combined with milk extracts containing sphinolipids, milk sphingolipids and/or metabolic derivatives of milk sphingolipids. Exemplary compositions include foods, dietary supplements, and pharmaceuticals adapted for human or veterinary consumption.

As used herein a “food” is a material consisting essentially of protein, carbohydrate, fat, and/or fibers which is used in the body of an organism to sustain growth, repair and vital processes and to furnish energy. Foods may also contain supplementary substances such as minerals, vitamins and condiments. See Merriam-Websters Collegiate Dictionary, 10^{th} Edition, 1993.

Sphingolipid-containing cocoa extracts, cocoa sphingolipids and metabolic derivatives thereof may be added to any food to provide sphingolipids (where there were none) or to enhance a pre-existing concentration of sphingolipids. The extracts, sphingolipids and metabolic derivatives thereof may be added to solid, liquid and semi-solid foods, including beverages. Preferably, they are added to foods containing cocoa ingredients (such as chocolate liquor, partially defatted cocoa solids, and/or fully defatted cocoa solids) to enhance the concentration of sphingolipids. Such foods and beverages are well known to those of skill in the art and may include Standard of Identity and Non-Standard of Identity confections, semi-sweet, sweet and milk chocolates, white chocolates, chocolate coatings and other foods such as cake mixes, ice creams, syrups, baked goods, spreads, beverages such as milk which may be chocolate/cocoa flavored. Examples of foods to which the cocoa sphingolipids may be added are as enumerated, for example, in International Patent Application No. PCT/US99/05414, published as WO99/45788.

In one embodiment, cocoa sphingolipids are added to foods containing cocoa polyphenols, more preferably cocoa procyanidins. These foods may contain enhanced levels of polyphenols in comparison to traditional cocoa-containing foods, which enhanced levels may be obtained, for example, by manipulating the processing of the cocoa bean into cocoa ingredient and/or by manipulating the formulating of the foods, and/or by adding a cocoa extract containing cocoa procyanidins, separated cocoa procyanidin monomers or oligomers, or synthetic procyanidins to conventionally made foods or beverages. Cocoa polyphenols and cocoa extracts containing cocoa polyphenols may be prepared as is known in the art and as described, for example, in U.S. Pat. Nos. 5,554,645 and 6,015,913; and International Patent Applications Nos. PCT/US96/04497 (published as WO97/36597), PCT/US97/15893 (published as WO 98/09533) and PCT/US99/05414 (published as WO 99/45788). The preparation of the synthetic procyanidins is described in the International Application No. PCT/US98/21392 (published as WO99/19319). The compositions of the invention may also contain L-arginine, prepared as described in International Patent Application No. PCT/US99/05545 (published as WO99/45797).

A “dietary supplement” is a product (other than tobacco) that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract or combination of these ingredients. See Merriam-Websters Collegiate Dictionary, 10^{th} Edition, 1993. The dietary supplement of the invention contains sphingolipid-containing cocoa extract, a cocoa sphingolipid, a metabolic derivative thereof, or a mixture thereof. It may also contain any of the ingredients discussed above. The dietary supplements may be manufactured using any methods known in the art and may be in solid, semi-solid or liquid form and any known dosage form, such as for example tablet, capsule, granule, powder and tincture.

As used herein, a “pharmaceutical” is a medicinal drug. See Merriam-Websters Collegiate Dictionary, 10^{th} Edition, 1993. The pharmaceuticals of the invention comprises an effective amount of a sphingolipid-containing cocoa extract, a cocoa sphingolipid, a metabolic derivative or any mixture thereof and a pharmaceutically acceptable carrier, diluent or excipient.

Suitable doses of pharmaceuticals containing sphingolipid-containing cocoa extract, cocoa sphingolipids, metabolic derivative or any mixture thereof will depend upon the particular medical application, the severity of the disease, the weight of the individual, the age of the individual, the sphingolipid half-life in circulation, and can be determined by a person of skill in the art. The number of doses, daily dosage and a course of treatment may be adjusted to fit each individual without undue experimentation. An effective amount of a cocoa sphingolipid can be determined using art-recognized methods, such as by establishing dose-response curves in suitable animal models, for example CF1 mouse as a model for colon cancer, and then extrapolating to humans and other mammals; by extrapolating from in vitro data; or by determining the effectiveness in clinical trials.

Pharmaceuticals containing the sphingolipid-containing extracts, cocoa sphingolipids or their metabolic derivatives may be administered in a variety of ways such as orally, nasally, rectally, intravenously, parenterally and topically. Dosage forms adapted for each administration type are also within the scope of the invention. Suitable pharmaceutically acceptable carriers, diluents, or excipients are generally known in the art and can be determined readily by a person skilled in the art.

The pharmaceuticals may be prepared in a variety of dosage forms such as tablets, capsules, bulk or unit dose powders or granules; within liposomes (prepared as described, for example, in U.S. Pat. No. 5,681,589 and U.S. Pat. No. 5,677,337 to Wei et al.); or they may be formulated into solutions, emulsions (prepared as described, for example, in U.S. Pat. No. 5,677,341), suspensions, ointments, pastes, creams, gels, foams or jellies. Sustained-release dosage forms are also within the scope of the invention and may be prepared as described in U.S. Pat. No. 5,994,492 to Graham et al., U.S. Pat. No. 5,993,843 to Sakurada et al., U.S. Pat. No. 5,989,586 to Hsu et al., U.S. Pat. No. 5,024,843 to Kuczynski et al.; U.S. Pat. No. 5,091,190 to Kuczynski et al; U.S. Pat. No. 5,082,668 to Wong et al.; U.S. Pat. No. 4,612,008 to Wong et al.; and U.S. Pat. No. 4,327,725 to Cortese et al. Parenteral dosage forms include, for example, solutions and suspensions. They may contain any of a variety of art-recognized excipients, diluents, fillers, etc. Such subsidiary ingredients include disintegrants, binders, lubricants, surfactants, emulsifiers, buffers, moisturizers, solubilizers and preservatives. These dosage forms are well known in the art and a person of skill can prepare the appropriate formulation comprising the sphingolipid-containing cocoa extract, isolated cocoa sphingolipid and/or metabolic derivative thereof using methods known in the art and described for example in “Goodman & Gilman's, The Pharmaceutical Basis of Therapeutics” (6th ed., Goodman et al., eds., MacMillan Publ. Co., NY, 1989).

Further within the scope of the invention is a package comprising a food, a dietary supplement or a pharmaceutical, and a label indicating an enhanced content of cocoa sphingolipids or a metabolic derivative thereof or indicating the beneficial properties of these compounds and, optionally, instructions for use. As used herein, the beneficial properties are: anti-neoplastic activity, altering the activity of protein kinases (including protein kinase C), inducing apoptosis or cellular differentiation, altering cell growth, mediating actions of tumor necrosis factor α, interleukin 1β, nerve growth factor, and 1α,25-dihydroxyvitamin D₃, inhibiting retinoblastoma protein dephosphorylation, activating phospholipase C/inhibiting phosphatidic acid phosphatase, altering diacylglycerol and cholesterol metabolism, and mobilizing Ca2⁺ from intracellular stores.

The invention further relates to methods of altering cell proliferation, for example, inhibiting cell proliferation or reducing the rate of proliferation. The inhibition need not be complete to be useful. Preferably, the method is used to inhibit proliferation of cancerous cells, for example, colon cancer cells or any gastrointestinal cancer cells. Most preferably, the method is used to alter cell proliferation of colon cancer cells. The method comprises the step of exposing animal or human cells to at least an effective amount of sphingolipid-containing cocoa extract, cocoa sphingolipid and/or a metabolic product thereof. The method further comprises the step of monitoring cell growth. An “effective amount” means that amount of cocoa extract, cocoa sphingolipid, metabolic derivative, or any combination thereof, that results in a measurable change in cell proliferation, the change being measured using methods known in the art. For example, an amount effective may be from about 0.005% of diet wt/wt to about 2%, more preferably from about 0.025% to about 2%, and most preferably from about 0.1% to about 2%, or about 10% of these values when administered directly to the cells. However, the cocoa extract, cocoa sphingolipid and/or a metabolic products thereof can be administered in excess of the effective amount. The cells may be exposed to the extracts and compounds for a prolonged period of time as determined by a person skilled in the art, for example, until the cell proliferation is completely inhibited. The method may be used alone or in combination with other known methods for reducing cell proliferation.

In another embodiment, the invention relates to a method of altering cell differentiation (inducing it or inhibiting it) or inducing apoptosis. The method may be practiced as described above by administering at least an amount effective to alter cell differentiation or induce apoptosis. These amounts equal the amounts described for the method of altering cell proliferation.

Another embodiment includes a method of treating colon cancer by administering to a human or an animal at least an effective amount of the cocoa extract, cocoa sphingolipid and/or a metabolic derivative thereof. The method further comprises the step of monitoring the progression of colon cancer, for example, by doing biopsies. The “effective amount” to treat colon cancer is that amount of the cocoa extract, cocoa sphingolipid and/or metabolic derivative thereof that results in at least a reduction in the disease progression rate as ascertained by a skilled professional. For example, an effective amount may be from about 0.005% of diet wt/wt to about 2%, more preferably from about 0.025% to about 2%, and most preferably, from about 0.1% to about 2%. However, the cocoa extract, cocoa sphingolipid and/or a metabolic derivative thereof can be administered in excess of the effective amount. They may be administered orally, buccally (i.e., directly into the stomach) or rectally in any form known to a person of skill in the art, for example through food, as a dietary supplement and as a pharmaceutical preparation. The administration may be continued for a prolonged period of time as determined by a person of skill in the art, for example until the cancer has been eliminated or the tumor progression from benign to malignant stage prevented. The method may be used alone or in combination with other known methods for altering cell proliferation. It may be used in individuals at early stages and at later stages of colon carcinogenesis. The method may also be used preventively.

Also included are methods for preventing abnormal cell proliferation and colon carcinogenesis by preventive exposure to or intake of the sphingolipid-containing cocoa extract, cocoa sphingolipid, and/or metabolic product thereof.

The invention is further described by the following non-limiting examples.

### EXAMPLES

### Example 1

Isolation and Purification of Cocoa Sphingolipids

Cocoa sphingolipids were extracted and purified from cocoa beans according to the following procedure.

About 16 g (16.409 g) of fermented West African cocoa beans were crushed in a Wiley mill or with a mortar and pestle and placed in a 125 ml Erlymnyer flask. Twenty ml of butanol (BuOH) were added to the crushed beans and the contents were boiled while stirring for 10 min at setting 3 on a hot plate. The liquid was decanted into a round-bottom (“RB”) flask and saved. The slurry remaining in the Erlynmyer flask was centrifuged to recover the supernatant. The supernatant was transferred to a round-bottom flask and the remaining solids were reextracted with BuOH as described above. Again, the liquid was decanted after boiling and the slurry was centrifuged to recover the supernatant. The liquid extracts from both butanol extractions, as well as the two supernatants, were all combined and saved.

The solids remaining after the above butanol extraction were then extracted with a mixture of CHCl₃ (chloroform): CH₃OH (methanol): H₂O (water) (28 ml total, 1:2:0.8 ratio) as described by M. Kates (Chapter 3: Lipid Extraction Procedures in Techniques of Lipidology; p. 105, 1986). The slurry was centrifuged and the supernatant containing cocoa extract was collected and combined with BuOH extracts in an RB flask. The solids were reextracted with chloroform:methanol:water, centrifuged, and the supernatant was combined with the rest of the cocoa extract in the RB flask. The extract was then dried in a rotary evaporator. It should be noted that BuOH may not be easy to remove by drying due to its boiling point of 117° C. at atmospheric pressure. Thus, either a rotary pump under partial pressure or high temperature is required to facilitate drying in this step.

One ml of methanol (MeOH) and two ml of hexane were mixed and added to the above dried cocoa extract, the contents were mixed to reconstitute the extract, and the solution was decanted to a screw cap test tube. This procedure was repeated twice. The contents of screw cap test tube were then centrifuged to separate the methanol and hexane layers. The hexane layer containing non-polar lipids such as fatty acids was collected and saved in a screw cap test tube. Two ml hexane was added to the methanol layer, which contained the cocoa sphingolipids, and the mixture was recentrifuged to ensure that all non-polar lipids were removed. The hexane layer was collected and combined with the previous hexane layer. The methanol layer containing cocoa sphingolipids was saved. The combined hexane layers were then extracted once with 2 ml MeOH to ensure that all sphingolipids were recovered. The methanol layer was collected and combined with the prior methanol layer and dried in a rotary evaporator. The hexane layer was discarded.

The base hydrolysis was carried out by reconstituting the dried fraction in a test tube with MeOH to a final volume of 4 ml. To this, 50 ml of 2N KOH in MeOH was added for every 1 ml of extract (total 200 ml), and the test tube was placed in a shaking water bath at 37° C. for 90 min to perform base hydrolysis. The base was then neutralized with an equal volume of 0.5N HCl in MeOH; and the solution was centrifuged and the supernatant transferred into an R.B. flask and dried in a rotary evaporator.

Two ml chloroform (CHCl₃) and 1 ml water were added to the dried extract, decanted to a screw cap test tube, and this procedure was repeated twice. The solutions were collected in a screw cap tube, the tube was then centrifuged to remove water, which water was then back extracted with 2 ml chloroform. The chloroform layers were combined and dried in a rotary evaporator. This extract was then used for Mass Spectrometry (MS) analysis as described below.

The purity of the cocoa sphingolipids at various stages of purification was checked by thin layer chromatography using silica gel 60 plates and CHCL₃/CH₃OH/CH₃COOH/H₂O (85:15:15:3, v/v) as the developing solvent.

MS analysis may be performed as is generally known in the art, for example, as described in Adams & Ann, Mass Spectrometry Reviews, 1993, 12: 51-85; Ann & Adams, Analytical. Chem., 1993, 65:7; and Gu et al., Analytical Biochem., 1997, 244:347-56. This study characterized the molecular species of GlcCer from cocoa by tandem mass spectrometry. A PE Sciex 3000 triple mass spec was used. The conditions for the analysis in the positive ion mode included: ionspray voltage 5000; orifice voltage 50-100; ring (skimmer) voltage 200-300; collision energy for fragmentation OR1—11V and OR2—50-70V.

Precursor ion scans of the molecularly distinctive product ions of m/z 262 revealed that cocoa GlcCer was comprised primarily of ceramide with 4, 8-sphingadiene (d18:2^{Δ4},^{Δ8}) and α-hydroxy fatty acids h16:0, h18:0, h20:0 and h24:0 (25.1, 41.5, 14.9, 2.7, and 1.0% relative abundance, respectively); the remainder had the same backbone with c16:0, c18:0, c20:0 fatty acids (5.9, 2.4, and 0.6% relative abundance, respectively). A small amount of GlcCer was detected which consisted of ceramide with 4-hydroxy-8-sphingenine (t18:1^{Δ8}) and α-hydroxy fatty acids h22:0, h24:0, and h26:0 (3.0, 2.7, and 0.4% relative abundance, respectively). These backbones differ from those of mammalian sphingolipids, which often have a Δ4-double bond (but rarely a Δ8-double bond), and have α-hydroxy-fatty acids in only some cases.

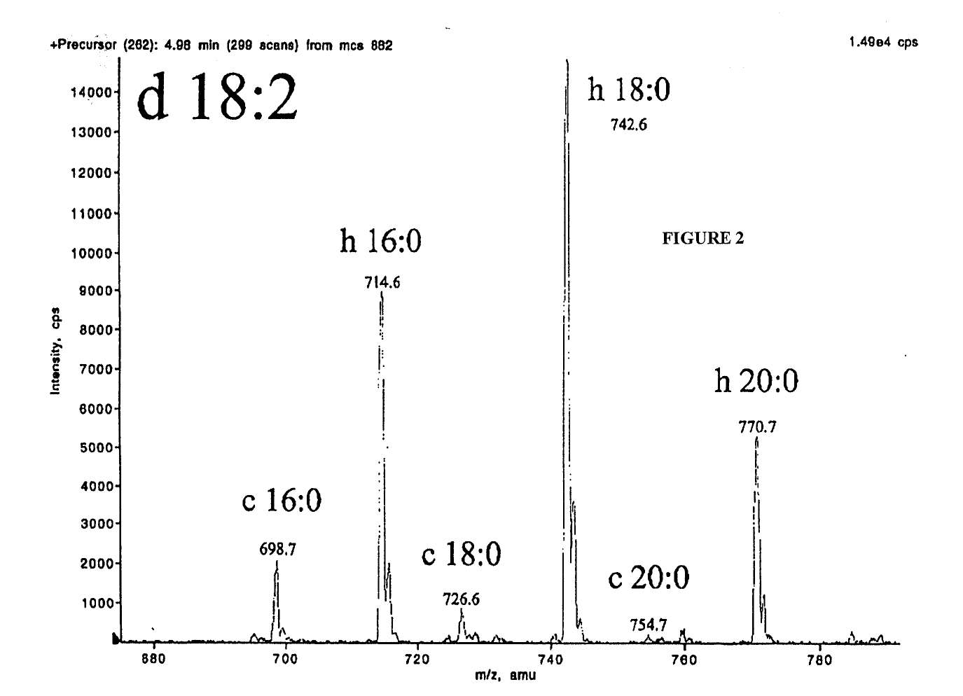

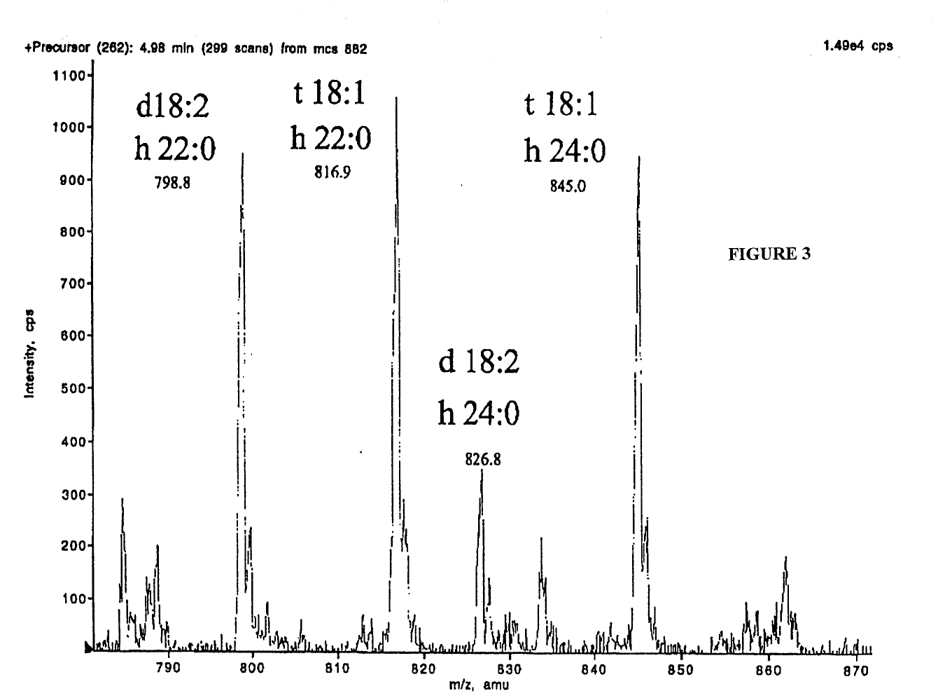

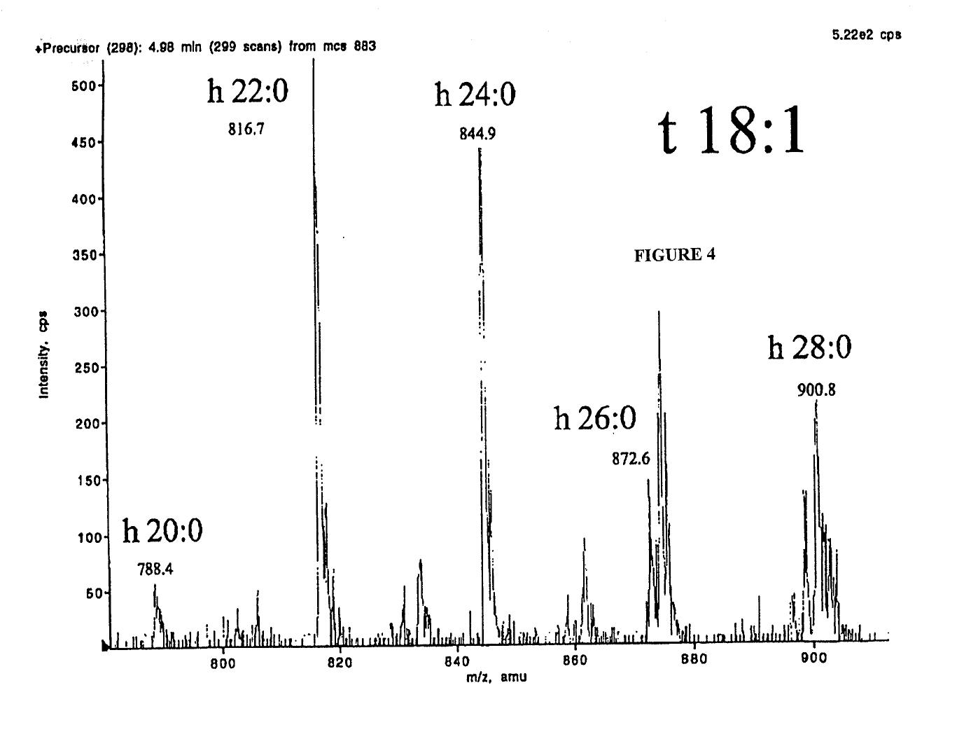

MS spectra of the above identified cocoa GIcCer with mass and structural assignments are represented in FIGS. 2-4.

### Example 2

Effect of Cocoa Sphingolipids on Cancer Cells in Vitro

An in vitro assay relying on tritiated thymidine incorporation is used to ascertain the effect of cocoa extracts, GlcCer and metabolic derivatives on cell proliferation. Briefly, tumor cells are seeded in flat bottom 96 well plates (Corning, N.Y.) at a concentration of 2×10⁴ cells per well. Four cancer cells lines are examined, HT29, a human colon cancer cell line, COLO-205, a human colon cancer line (ATCC No. CCL 222); Lu-65, a lung cancer cell line (T. Yamada et al., Jpn. J. Cancer Res., 76, 967-976 (1985); and MKN-74, a gastric cancer cell line (Motoyama et al., Acta Med. Biol., 27, 49-63 (1979). The cells are cultured for 2 days in DMEM containing various concentrations of cocoa sphingolipids or extracts containing sphingolipids which are added as a PBS solution. The medium is then supplemented with tritiated thymidine at a concentration of 0.5 μCi per well. Following a six hour incubation the cells are collected using the PHYD Cell Harvester (Cambridge Technology, Cambridge, Mass.) and amounts of incorporated radioactivity are determined after adding a suitable cell lysing agent and scintillation cocktail, such as ScintiVerse BD (Fisher Scientific, Fairlawn, Calif.).

### Example 3

Effect of Cocoa Sphingolipids on Colon Cancer in Vivo

The sphingolipids are tested using a colon cancer animal model according to the following procedure.

Female CF1 mice (Charles Rivers Laboratories, Portage, Mich.), five weeks of age and virus antibody free, are group housed on hardwood chip bedding in microisolator cages. Mice are maintained in a relative humidity of 50-60%, a temperature of 23±2° C., and a 12:12-hour light:dark cycle. The mice are fed Purina Rodent Chow 5001 (Ralston Purina, St. Louis, Mo.) on arrival and have free access to water. The animals are weighed weekly and monitored closely for signs of illness.

After initial acclimatization for one week, the mice are fed a semi-purified AIN 76A diet (PMI Feeds, Richmond, Ind.) that is essentially sphingolipid-free as determined by high-performance liquid chromatography which is used as the control diet (American Institute of Nutrition, J Nutr 107, 1340-1348, 1977). The mice are injected intraperitoneally with 0.5 ml of DMH HCl (30 mg/kg body wt) in 1 mM EDTA (Sigma Chemical, St. Louis, Mo.) once per week for six weeks. Starting one week after the last injection, the mice are randomly divided into six animals per group and are fed the AIN 76A diet alone or supplemented with cocoa extract containing GlcCers, individual GIcCers, metabolic derivatives or mixtures thereof. The amount of GlcCer in the diet is confirmed by thin-layer and high-performance liquid chromatography, as described by Merrill, et al, Anal Chem 171, 373-381, 1988.

After four weeks of feeding, the mice are killed by CO₂ asphyxiation, and the colons are removed, flushed with cold 0.05 M tris(hydroxymethyl)aminomethane HCl, pH 7.6 at 4° C., opened longitudinally, fixed flat overnight in 10% neutral buffered formalin, and stained with 0.2% methylene blue for 20-40 minutes. Foci of aberrant crypts are scored by light microscopy at x40 or x100 magnification, as described by Bird, RP, Cancer Lett 37, 147-151, 1987.

All statistical analyses are executed using the Instat software (version 1, Instat, San Diego, Calif.). The weights and number of ACF are evaluated by Student's t-test (analysis of variance). The correlation of animal weight and number of ACF is evaluated by regression analysis. Differences are considered significant at p<0.05.

A long term study is also conducted using the above described protocol except that feeding is continued for 40 weeks before the mice are sacrificed.

What is claimed is:

1. A sphingolipid-containing cocoa extract which is substantially free of cocoa polyphenols.

2. The sphingolipid-containing cocoa extract of claim 1 comprising a sphingolipid selected from the group consisting of d18:2^{Δ4,Δ8}/c16:0; d18:2^{Δ,Δ8}/c18:0; d18:2^{Δ4,Δ8}/c20:0; d_{18:2}^{Δ4,Δ8}/h16:0; d18:2^{Δ4,Δ8}/h18:0; d18:2^{Δ4,Δ8}/h20:0; d18:2^{Δ4,Δ8}/h_{22:0}; d18:2^{Δ4,Δ8}/24:0; t18:1^{Δ4,Δ8}/h20:0; t18:1^{Δ8}/h22:0; t18:1^{Δ8}/h24:0; t18:1^{Δ8}/h26:0; t18:1^{Δ8}/h28:0 and any mixture thereof.

3. The sphingolipid-containing cocoa extract of claim 2 comprising a sphingolipid selected from the group consisting of d18:2/h18:0; d18:2/h16:0; t18:1/h22:0; t18:1/h24:0; d18:2/h16:0 and any mixture thereof.

4. The sphingolipid-containing cocoa extract of claim 3, wherein said sphingolipids are present in the following order of prevalence:
d18:2/h18:0>d18:2/h16:0=t18:1/h22:0=t18:1/h24:0>d18:2/h16:0.

5. The sphingolipid-containing cocoa extract obtained by extracting polar lipids from a cocoa material.

6. The sphingolipid-containing cocoa extract of claim 5, wherein said extract is purified.

7. The sphingolipid-containing cocoa extract of claim 6, wherein said extract is purified by removing non-polar lipids.

8. The sphingolipid-containing cocoa extract of claim 6, wherein said extract is purified by hydrolyzing the extract with a base, separating the hydrolyzed products, and recovering the purified extract.

9. The sphingolipid-containing cocoa extract of claim 6, wherein said extract is substantially pure.

10. A sphingolipid-containing cocoa extract which is prepared by a process comprising the steps of:
(i) extracting polar lipids from a cocoa material to obtain a crude sphingolipid-containing extract;
(ii) hydrolyzing said crude sphingolipid-containing extract with a base; and
(iii) removing the products of said base hydrolysis.

11. The sphingolipid-containing cocoa extract of claim 10, wherein said process further comprises the step of extracting non-polar lipids from the crude sphingolipid-containing extract prior to step (ii).

12. A sphingolipid-containing cocoa extract prepared by a process comprising the steps of:
(i) extracting a cocoa material with butanol to obtain a crude extract containing sphingolipids;
(ii) extracting the crude extract with hexane to obtain a hexane-purified cocoa extract;
(iii) hydrolyzing the hexane-purified cocoa extract with a base to obtain a base-hydrolyzed cocoa extract; and
(iv) extracting the base-hydrolyzed cocoa extract with chloroform to remove the products of base hydrolysis.

13. A sphingolipid selected from the group consisting of d81:2^{Δ4,Δ8}/c16:0; d18:2^{Δ4,Δ8}/c18:0; d18:2^{Δ4,Δ8}/c20:0; d_{18:2}^{Δ4,Δ8}/h16:0; d18:2^{Δ4,Δ8}/h18:0; d18:2^{Δ4,Δ8}/h20:0; d18:2^{Δ4,Δ8}/h22:0; d18:2^{Δ4,Δ8}/24:0; t18:1^{Δ8}/h20:0; t18:1^{Δ8}/h22:0; t18:1^{Δ8}/h24:0; t18:1^{Δ8}/h26:0; and t18:1^{Δ8}/h28:0.

14. A composition comprising the sphingolipid-containing cocoa extract of claim 1, a cocoa sphingolipid or a metabolic derivative thereof.

15. The composition of claim 14, further comprising milk, a sphingolipid-containing milk extract, a milk sphingolipid or a metabolic derivative thereof.

16. The composition of claim 14, wherein said composition is a foodstuff, a pharmaceutical or a dietary supplement.

17. The composition of claim 16, further comprising a cocoa polyphenol.

18. The composition of claim 17, wherein said polyphenol is procyanidin.

19. The composition of claim 16, wherein said foodstuff is selected from the group consisting of a chocolate confectionery, a chocolate beverage, and a chocolate-containing spread.

20. The composition of claim 19, wherein said chocolate confectionery is a dark chocolate, a white chocolate, a milk chocolate or a candy comprising a chocolate coating.

21. The composition of claim 19, wherein said chocolate beverage is chocolate milk.

22. A package comprising the composition of claim 14 and a label or instruction for use.

23. A method for extracting and purifying sphigolipids from a sphingolipid-containing material comprising the steps of:
(i) extracting polar lipids from a sphingolipid-containing material to obtain a crude extract; and
(ii) extracting the crude extract with hexane to obtain a hexane-purified extract.

24. The method of claim 23, further comprising the steps of:
(i) hydrolyzing the hexane-purified extract with a base to obtain a base-hydrolyzed extract; and
(ii) removing the hydrolyzed by-products to provide a base-hydrolyzed extract.

25. A method of altering cell proliferation by contacting the cell with the sphingolipid-containing cocoa extract, a cocoa sphingolipid or a metabolic derivative thereof.

26. The method of claim 25, wherein the cell proliferation is reduced.

27. A method of treating cancer by administering to a mammal a composition comprising a sphingolipid-containing cocoa extract, a cocoa sphingolipid or a metabolic derivative thereof.

28. The method of claim 27, wherein said cancer is colon cancer.
